(19) United States
(12) Patent Application Publication
(10) Pub. No.: US 2009/0269354 A1
(43) Pub. Date: Oct. 29, 2009

(54) QUINAZOLINE DERIVATIVES AND METHODS OF TREATMENT

(75) Inventors: Craig E. Masse, Cambridge, MA (US); Roger Tung, Lexington, MA (US)

Correspondence Address: EDWARDS ANGELL PALMER & DODGE LLP, P.O. BOX 55874, BOSTON, MA, 02205, US

(73) Assignee: Concert Pharmaceuticals, Inc., Lexington, MA (US)

(21) Appl. No.: 12/413,510

(22) Filed: Mar. 27, 2009

Related U.S. Application Data
(60) Provisional application No. 61/157,549, filed on Mar. 4, 2009.
(60) Provisional application No. 61/040,647, filed on Mar. 28, 2008.

(51) Int. Cl. A61K 39/395; C07D 239/94; A61K 31/517; A61P 35/00

(52) U.S. Cl. 424/145.1; 544/293; 514/266.4

(57) ABSTRACT

This invention relates to novel quinazoline derivatives, and their pharmaceutically acceptable salts. The invention also provides compositions comprising a compound of this invention and the use of such compositions in methods of treating diseases and conditions beneficially treated by inhibiting cell surface tyrosine receptor kinases.

### RELATED APPLICATIONS

This application claims the benefit of U.S. Provisional Patent Application Nos. 61/157,549, filed Mar. 4, 2009, and 61/040,647, filed Mar. 28, 2008. The entire contents of each of these applications are incorporated herein by reference.

### TECHNICAL FIELD OF THE INVENTION

This invention relates to novel quinazoline derivatives and their pharmaceutically acceptable salts. The invention also provides compositions comprising a compound of this invention and the use of such compositions in methods of treating diseases and conditions beneficially treated by inhibiting cell surface tyrosine receptor kinases.

Quinazoline derivatives which bear at the 4-position an anilino substituent and which also bear an alkoxy substituent at the 7-position and an alkoxy substituent at the 6-position, are disclosed inter alia in U.S. Pat. No. 5,770,599, U.S. Pat. No. 5,747,498, EP 1,110,953, EP 817,775, and U.S. Pat. No. 6,476,040. One of those derivatives, erlotinib, is known chemically as [6,7-Bis-(2-methoxy-ethoxy)-quinazolin-4-yl]-(3-ethynyl-phenyl)-amine and as N-(3-ethynylphenyl)-6,7-bis(2-methoxyethoxy)quinazolin-4-amine.

Erlotinib is an inhibitor of tyrosine kinases, particularly EGF receptor tyrosine kinases. Erlotinib has been approved in the United States in and in Europe for the treatment of locally advanced or metastatic non-small cell lung cancer (NSCLC) after failure of at least one prior chemotherapy regimen. Erlotinib is also approved in the United States in combination with gemcitabine, for the treatment of metastatic pancreatic cancer. Clinical trials are ongoing investigating the use of erlotinib alone or in combination with other agents for the treatment of a variety of cancers, including non-small cell lung cancer, ovarian cancer, colorectal cancer, head and neck cancer, brain cancer, bladder cancer, sarcoma, prostate cancer, melanoma, cervical cancer, solid tumors, astrocytoma, breast cancer, pancreatic cancer, glioblastoma multiform, renal cancer, digestive/gastrointestinal cancer, liver cancer, gynecological cancers, CNS tumors, thymoma and gastric cancer. Erlotinib is also thought to be useful in the treatment of benign hyperplasia of the skin (psoriasis) or prostate (BPH).

The most frequently reported adverse events occurring in patients dosed with erlotinib include, but are not limited to, rash, diarrhea, anorexia, fatigue, dyspepsia, nausea, infection, stomatitis, pruritus, dry skin, conjunctivitis, pyrexia, depression, cough, headache, and liver function test abnormalities. (See FDA label for Tarceva, accessed at http://www.fda.gov/cder/foi/label/2007/021743 s007 lbl.pdf).

The efficacy of tyrosine kinase inhibitors, such as erlotinib, may be related to some extent to whether or not the patient receiving the drug is or has ever been a smoker. This may be due in part to more rapid metabolism of the tyrosine kinase inhibitor by smokers and ex-smokers, as compared to non-smokers. Lynch T J et al, N Engl J Med 2004, 350:2129-2139; Pao W et al, Proc Natl Acad Sci USA 2004, 101:13306-13311; Marchetti A et al, J Clin Oncol 2005, 23:857-865; Shigematsu H et al, J Natl Cancer Inst 2005, 97:339-346; and Pham D et al, J Clin Oncol 2006, 24: 1700-1704.

Despite the beneficial activities of erlotinib, there is a continuing need for new compounds to treat the aforementioned diseases and conditions.

### DEFINITIONS

The terms “ameliorate” and “treat” are used interchangeably and both mean decrease, suppress, attenuate, diminish, arrest, or stabilize the development or progression of a disease (e.g., a disease or disorder delineated herein), lessen the severity of the disease or improve the symptoms associated with the disease.

By “disease” is meant any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of erlotinib will inherently contain small amounts of deuterated isotopologues. The concentration of naturally abundant stable hydrogen isotopes, notwithstanding this variation, is small and immaterial with respect to the degree of stable isotopic substitution of compounds of this invention. See, for instance, Wada, E et al., Seikagaku, 1994, 66: 15; Ganes, L Z et al., Comp Biochem Physiol Mol Integr Physiol, 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to species that differ from a specific compound of this invention only in the isotopic composition of its molecules or ions.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

A compound of this invention may exist in salt form. A salt of a compound is formed between an acid and a basic group of the compound, such as an amino functional group, or a base and an acidic group of the compound, such as a carboxyl functional group. According to another preferred embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

The compounds of the present invention may contain one or more asymmetric carbon atoms. As such, a compound of this invention can exist as the individual stereoisomers (enantiomers or diastereomers) as well a mixture of stereoisomers. Accordingly, a compound of the present invention will include not only a stereoisomeric mixture, but also individual respective stereoisomers substantially free from one another stereoisomers. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present Methods of obtaining or synthesizing diastereomers are well known in the art and may be applied as practicable to final compounds or to starting material or intermediates. Other embodiments are those wherein the compound is an isolated compound. The term “at least X % enantiomerically enriched” as used herein means that at least X % of the compound is a single enantiomeric form, wherein X is a number between 0 and 100, inclusive.

The term “stable compounds”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

A “metabolically labile protecting group” is chemical moiety that is cleaved in vivo by one or more of an organism's naturally occurring enzyme.

“D” refers to deuterium. “Stereoisomer” refers to both enantiomers and diastereomers. “Tert” and “t-” each refer to tertiary. “US” refers to the United States of America. “FDA” refers to Food and Drug Administration. “NDA” refers to New Drug Application. “rt” refers to room temperature.

The term “smoker” as used herein means a human that has a smoking history of more than 15 pack-years and who has smoked in the past 25 years. The term “non-smoker” as used herein means a human that has a smoking history of 15 pack years or less, or who has not smoked for over 25 years. A “pack-year” is calculated by multiplying the number of cigarettes smoked per day by the number of years smoked and dividing that product by 20 (see http://www.cancer.gov/Templates/db_alpha.aspx?CdrID=306510).

Throughout this specification, reference to “each Y” includes, independently, all “Y” groups (e.g., Y^{1a}, Y^{1b}, Y^{1c}, Y^{2a}, Y^{2b}, and Y^{2c}) where applicable. Likewise, reference to “each Z” includes, independently, all “Z” groups (e.g., Z^{1a}, Z^{1b}, Z^{2a}, and Z^{2b}) where applicable; and reference to “each X” includes, independently, all “X” groups (e.g., X^{1a}, X^{1b}, X^{2a}, and X^{2b}) where applicable.

### Therapeutic Compounds

In one embodiment, the present invention provides a compound of Formula Q:

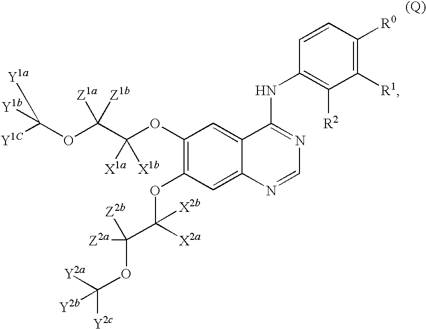

or a salt thereof, wherein:

each X, each Y, and each Z is independently selected from hydrogen and deuterium;

R⁰ is selected from hydrogen, halo, —OH, —OCD₃, and —OCH₃;

R¹ is selected from —C≡CH, and —C≡CD; and

R² is selected from hydrogen and fluoro;

provided that at least one of X, Y or Z is deuterium; and

further provided that when each of R⁰ and R² is hydrogen, at least one X is deuterium; and

further provided that when each of R⁰ and R² is hydrogen, R¹ is —C≡CH, and each X and each Z is deuterium, then at least one Y is deuterium.

In one embodiment of a compound of Formula Q, each Y is the same, each Z is the same, and each X is the same. In one aspect of this embodiment, each Y is deuterium and each Z is deuterium. In a further aspect of this embodiment, each X is deuterium. In still another aspect each Y is deuterium, each Z is deuterium and each X is hydrogen.

Another embodiment provides a compound of Formula Q, wherein each Y¹ is the same, each Y² is the same, each Z¹ is the same, each Z² is the same, each X¹ is the same and each X² is the same.

Another embodiment provides a compound of Formula Q, wherein each Y¹ is deuterium and each Z¹ is deuterium. In a further aspect of this embodiment, each X¹ is deuterium.

In still another embodiment each Y² is deuterium and each Z² is deuterium. In a further aspect of this embodiment, each X² is deuterium.

In still another embodiment, R⁰ is halo. In one aspect of this embodiment R² is hydrogen. In a further aspect of this embodiment, each Y is deuterium and each Z is deuterium. In still a further aspect of this embodiment each Y is deuterium, each Z is deuterium and each X is hydrogen.

In still another embodiment, each Y¹ is deuterium, each Y² is deuterium, each Z¹ is deuterium, each Z² is deuterium, each X¹ is hydrogen, each X² is hydrogen, R¹ is —C≡CH, R² is hydrogen, and R⁰ is Br or F.

Another embodiment of Formula Q provides a compound of Formula A:

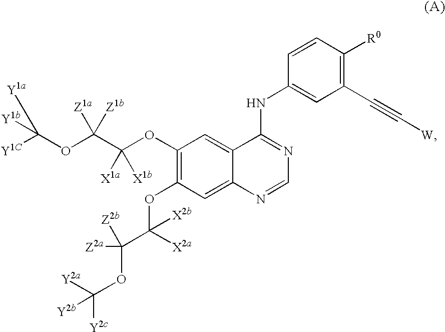

or a salt thereof, wherein:

each X, each Y, each Z and W is independently selected from hydrogen and deuterium; and

R⁰ is hydrogen, OH, F, OCD₃, or OCH₃;

provided that at least one of X, Y or Z is deuterium; and

provided that if R⁰ is hydrogen, then at least one X is deuterium; and

further provided that if R⁰ is hydrogen, and W is hydrogen, and each X and each Z is deuterium, then at least one Y is deuterium.

In certain embodiments of Formula A:

a) R⁰ is not hydrogen, and at least one X, Y, Z or W is deuterium;

b) Each Y¹ is the same;

c) Each Y² iS the same;

d) Each Z¹ is the same;

e) Each Z² iS the same;

f) Each X¹ is the same; or

g) Each X² is the same.

In still other embodiments of Formula A:

h) Each Y¹ is deuterium;

i) Each Y² is deuterium;

j) Each Z¹ is deuterium;

k) Each Z² is deuterium;

l) Each X¹ is deuterium; or

m) Each X² is deuterium.

In a more specific embodiment, a compound of Formula A has the properties of two or more of a) through m), above.

In still another specific embodiment of Formula A, each Y is deuterium, each Z is deuterium and W is hydrogen.

In yet another embodiment of Formula A, each Y is the same, each X is the same, and each Z is the same. In one aspect of this embodiment, each Y is deuterium, each X is deuterium, each Z is deuterium and R⁰ is hydrogen, or OH. In yet another aspect of this embodiment, each Y is deuterium, each X is hydrogen, each Z is deuterium, and R⁰ is OH.

One embodiment of Formula A provides a compound of Formula I:

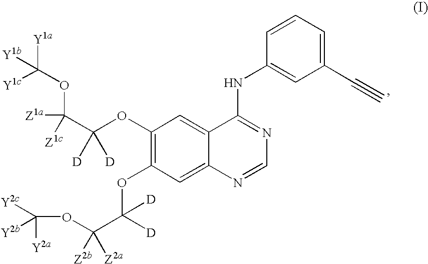

or a salt thereof, where Y and Z are as defined above; provided that when each Z is deuterium, then at least one Y is deuterium.

Examples of specific compounds of Formula Q include those delineated in Table 1 below.

TABLE 1

| | Each | Each | Each | Each | Each | Each | | | |
|---|---|---|---|---|---|---|---|---|---|
| Cmpd | Y¹ | Y² | Z¹ | Z² | X¹ | X² | R⁰ | R¹ | R² |
| 113 | D | D | D | D | H | H | Br | —C≡CH | H |
| 118 | D | D | D | D | H | H | F | —C≡CH | H |

Examples of specific compounds of Formula A include those delineated in Table 2 below.

TABLE 2

| | | | | Each | Each | Each | | |
|---|---|---|---|---|---|---|---|---|
| Cmpd | Each Y¹ | Each Y² | Each Z¹ | Z² | X¹ | X² | W | R⁰ |
| 120 | D | D | D | D | D | D | H | H |
| 121 | D | D | D | D | D | D | H | F |
| 122 | D | D | D | D | D | D | H | OCH₃ |
| 123 | D | D | D | D | D | D | H | OCD₃ |
| 124 | D | D | D | D | D | D | H | OH |
| 125 | D | D | D | D | D | H | H | H |
| 126 | D | D | D | D | D | H | H | F |
| 127 | D | D | D | D | D | H | H | OCH₃ |
| 128 | D | D | D | D | D | H | H | OCD₃ |
| 129 | D | D | D | D | D | H | H | OH |
| 130 | D | D | D | D | H | D | H | H |
| 131 | D | D | D | D | H | D | H | F |
| 132 | D | D | D | D | H | D | H | OCH₃ |
| 133 | D | D | D | D | H | D | H | OCD₃ |
| 134 | D | D | D | D | H | D | H | OH |

Other examples of specific compounds of Formula A include those set forth in Table 3, below.

TABLE 3

| | | | | Each | Each | Each | | |
|---|---|---|---|---|---|---|---|---|
| Cmpd | Each Y¹ | Each Y² | Each Z¹ | Z² | X¹ | X² | W | R⁰ |
| 135 | D | D | D | D | H | H | H | F |
| 136 | D | D | D | D | H | H | H | OCH₃ |
| 137 | D | D | D | D | H | H | H | OCD₃ |
| 138 | D | D | D | D | H | H | H | OH |

In another set of embodiments, any atom not designated as deuterium in any of the embodiments set forth above is present at its natural isotopic abundance.

In certain embodiments, the compound is Compound 120, 125, or 130.

In other embodiments, the compound is selected from one of:

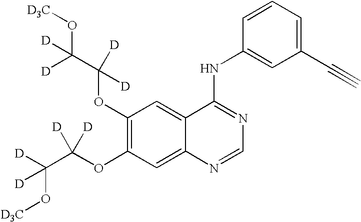

### Compound 120, and

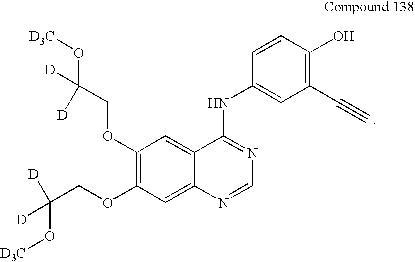

In another embodiment the compound is Compound 113:

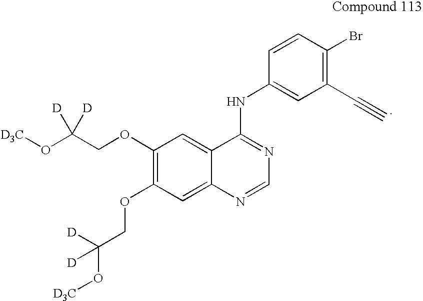

In another embodiment, the present invention provides a compound of Formula R:

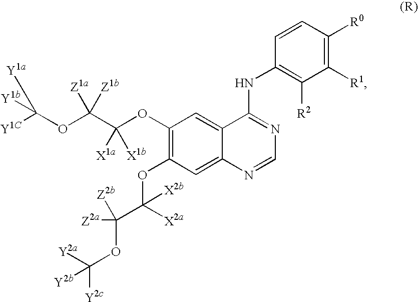

or a salt thereof, wherein:

each X, each Y, and each Z is independently selected from hydrogen and deuterium;

R⁰ is selected from hydrogen, halo, —OH, —OCD₃, and —OCH₃;

R¹ is selected from hydrogen, halo, and —CF₃; and

R² is selected from hydrogen and fluoro;

provided that at least one X, Y or Z is deuterium.

In one embodiment of a compound of Formula R, each Y is the same, each Z is the same, and each X is the same. In one aspect of this embodiment, each Y is deuterium and each Z is deuterium. In a further aspect of this embodiment, each X is deuterium. In still another aspect of this embodiment each Y is deuterium, each Z is deuterium and each X is hydrogen.

Another embodiment provides a compound of Formula R, wherein each Y¹ is the same, each Y² is the same, each Z¹ is the same, each Z² is the same, each X¹ is the same and each X² is the same.

Another embodiment provides a compound of Formula R, wherein each Y¹ is deuterium and each Z¹ is deuterium. In a further aspect of this embodiment, each X¹ is deuterium.

In still another embodiment, each Y² is deuterium and each Z² is deuterium. In a further aspect of this embodiment, each X² is deuterium.

In another embodiment of Formula R, R⁰ is selected from hydrogen and halo. In one aspect of this embodiment each Y is deuterium and each Z is deuterium. In a further aspect of this embodiment each Y is deuterium, each Z is deuterium and each X is hydrogen.

Examples of specific compounds of Formula R include those delineated in Table 4 below.

TABLE 4

| | | | Each | Each | Each | Each | | | |
|---|---|---|---|---|---|---|---|---|---|
| Cmpd | Each Y¹ | Each Y² | Z¹ | Z² | X¹ | X² | R⁰ | R¹ | R² |
| 105 | D | D | D | D | H | H | H | Br | H |
| 106 | D | D | D | D | H | H | H | Cl | H |
| 107 | D | D | D | D | H | H | H | F | H |
| 108 | D | D | D | D | H | H | H | CF₃ | H |
| 109 | D | D | D | D | H | H | Br | Br | H |
| 110 | D | D | D | D | H | H | Br | Cl | H |
| 111 | D | D | D | D | H | H | Br | F | H |
| 112 | D | D | D | D | H | H | Br | CF₃ | H |
| 114 | D | D | D | D | H | H | F | Br | H |
| 115 | D | D | D | D | H | H | F | Cl | H |
| 116 | D | D | D | D | H | H | F | F | H |
| 117 | D | D | D | D | H | H | F | CF₃ | H |
| 119 | D | D | D | D | H | H | Br | H | F |

In certain embodiments, the compound of Formula R is selected from any one of:

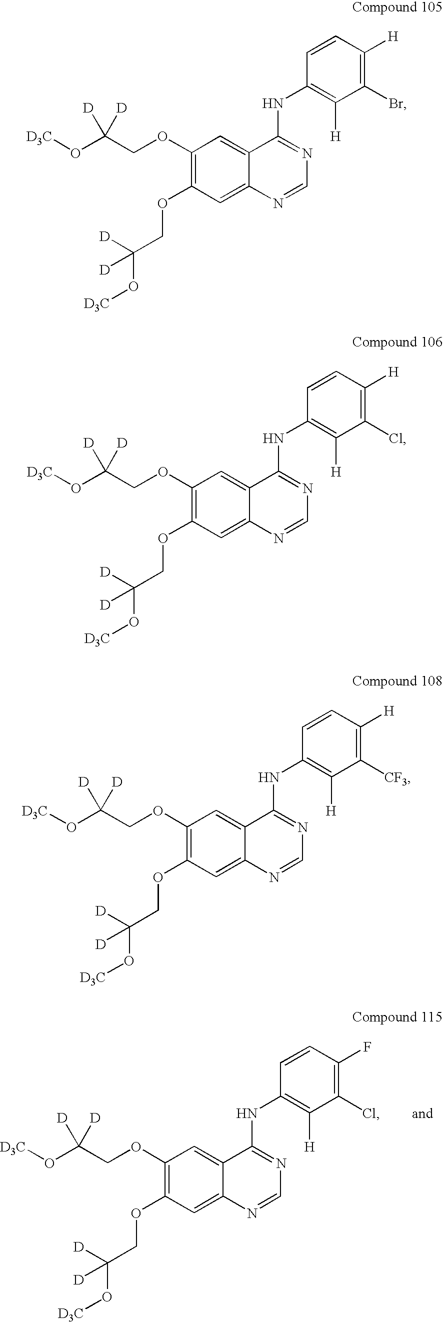

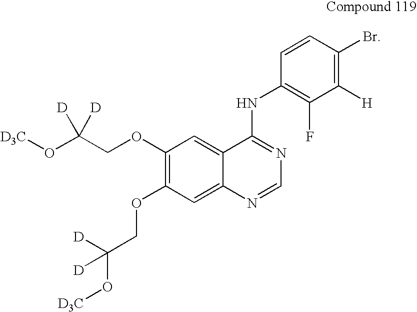

In another set of embodiments, any atom not designated as deuterium in any of the embodiments of Formulae Q, A, R or I, set forth above is present at its natural isotopic abundance.

The compounds of this invention may be made by synthetic chemists of ordinary skill. Relevant procedures and intermediates are disclosed, for instance, in U.S. Pat. No. 5,747,498, EP 1,110,953, EP 817,775, U.S. Pat. No. 6,900,221, U.S. Pat. No. 6,476,040 and PCT publication WO2007/060691. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

Convenient methods for producing compounds of Formulae Q, A, I and/or R are described in Schemes 1-5.

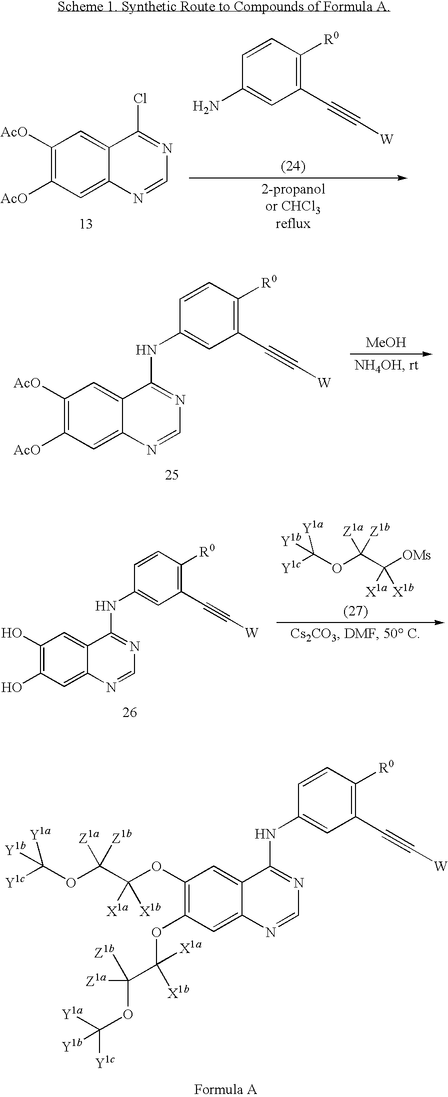

Scheme 1 depicts a general synthetic route for the preparation of a compound of Formula A. Chloroquinazoline 13 (prepared as described in Ramanadhan, J P et al., WO 2007060691A2) may be combined with an appropriately deuterated and optionally substituted ethynyl aniline 24 to form diacetylquinazolinamine 25. Deacetylation of diacetylquinazolinamine 25 with ammonium hydroxide and methanol provides the corresponding quinazolinamine 26, which then may be combined with an appropriately deuterated 2-methoxyethyl methane sulfonate 27 to form a compound of Formula A.

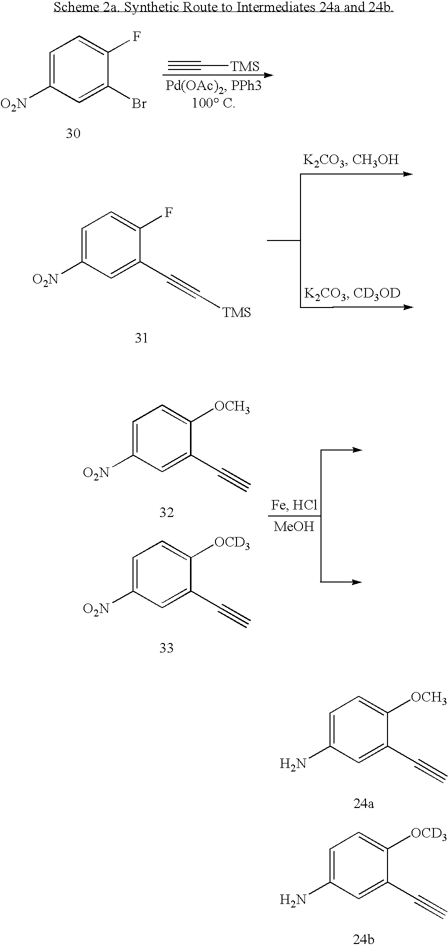

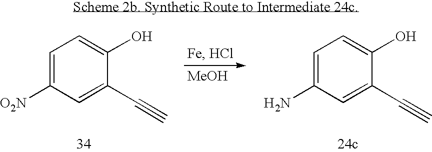

Schemes 2a and 2b show the synthesis of an appropriately deuterated and appropriately substituted ethynyl aniline intermediate 24. As shown in Scheme 2a, commercially available 3-bromo-4-fluoronitrobenzene 30 may be treated with ethynyltrimethylsilane under Sonogashira coupling conditions (palladium (II) acetate and triphenylphosphine) in anhydrous diethylamine according to the procedure described by Lau, K S Y et al., J Org Chem, 1981, 46: 2280-2286 to afford 2-fluoro-5-nitro-1-[(trimethylsilyl)ethynyl]benzene 31. Subsequent treatment of the trimethylsilyl-acetylene 31 with potassium carbonate in either methanol or d3-methanol affords 2-methoxy-5-nitrophenylacetylene 32 or 2-(methoxy-d₃)-5-nitrophenylacetylene 33, respectively. By this method, compound 33 may be prepared having at least 99% deuterium incorporation at the methoxy carbon. Finally, reduction of the nitro group with iron filings in the presence of hydrochloric acid using the procedures from the paper described above furnishes the 3-ethynyl-4-methoxyaniline 24a (wherein R is OCH₃ and W is H) and 3-ethynyl-4-(methoxy-d₃)aniline 24b (wherein R is OCD₃ and W is H), respectively.

According to the synthesis outlined in Scheme 2b, a methanolic solution of commercially available 2-hydroxy-5-nitrophenylacetylene (34) may be mixed with iron filings in the presence of hydrochloric acid according to the procedure described by Lau, K S Y et al., J Org Chem, 1981, 46:2280-2286 to afford the desired 3-ethynyl-4-hydroxyaniline 24c wherein R is OH and W is H.

4-Fluoro-3-(ethynyl)aniline, (24d), may be prepared according to the procedure detailed in J Org Chem, 1981, 2280-2296.

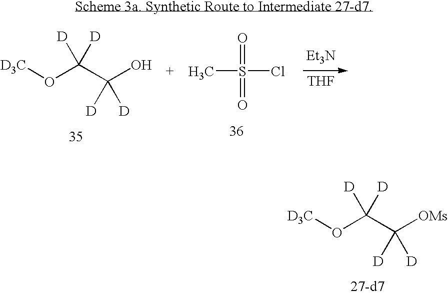

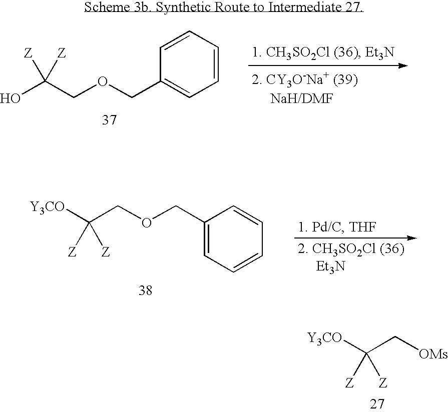

Schemes 3a and 3b depict the synthesis of various appropriately deuterated 2-methoxyethyl methane sulfonates 27. According to Scheme 3a, commercially available 2-(methoxy-d₃)-1,1,2,2-d₄-ethanol 35 may be combined with methanesulfonyl chloride 36 in the presence of triethylamine according to the procedure described in Japanese Patent Publication JP2002-293773A to afford the desired perdeuterated mesylate 27-d₇. The deuterated starting material, 2-(methoxy-d₃)-1,1,2,2-d₄-ethanol, 35, is available at 99 atom % D and thus compound 27-d7 may be prepared having >98% deuterium incorporation at the positions indicated (each X, each Y, and each Z). According to Scheme 3b, an appropriately deuterated 2-benzyloxyethanol 37 may be combined with methanesulfonyl chloride 36 in the presence of triethylamine and an appropriately deuterated sodium methoxide 39 to form the corresponding appropriately deuterated 2-methoxyethoxymethylbenzene 38. Examples of an appropriately deuterated 2-benzyloxyethanol 37 include commercially available 2-benzyloxyethanol 37 and 2-benzyloxy-(1,1-d₂-ethanol) 37-d2 which may be synthesized according to the procedure described in J Label Comp Radiopharm, 1989, 27(2): 199-216 using LiAlD₄ (98 atom % D). An appropriately deuterated sodium methoxide 39 may be pre-formed by reduction of an appropriately deuterated methanol with sodium hydride. The diether 38 may be reduced over Pd/C and coupled with methanesulfonyl chloride 36 in triethylamine to produce the appropriately deuterated 2-methoxyethyl methane sulfonate reagent 27.

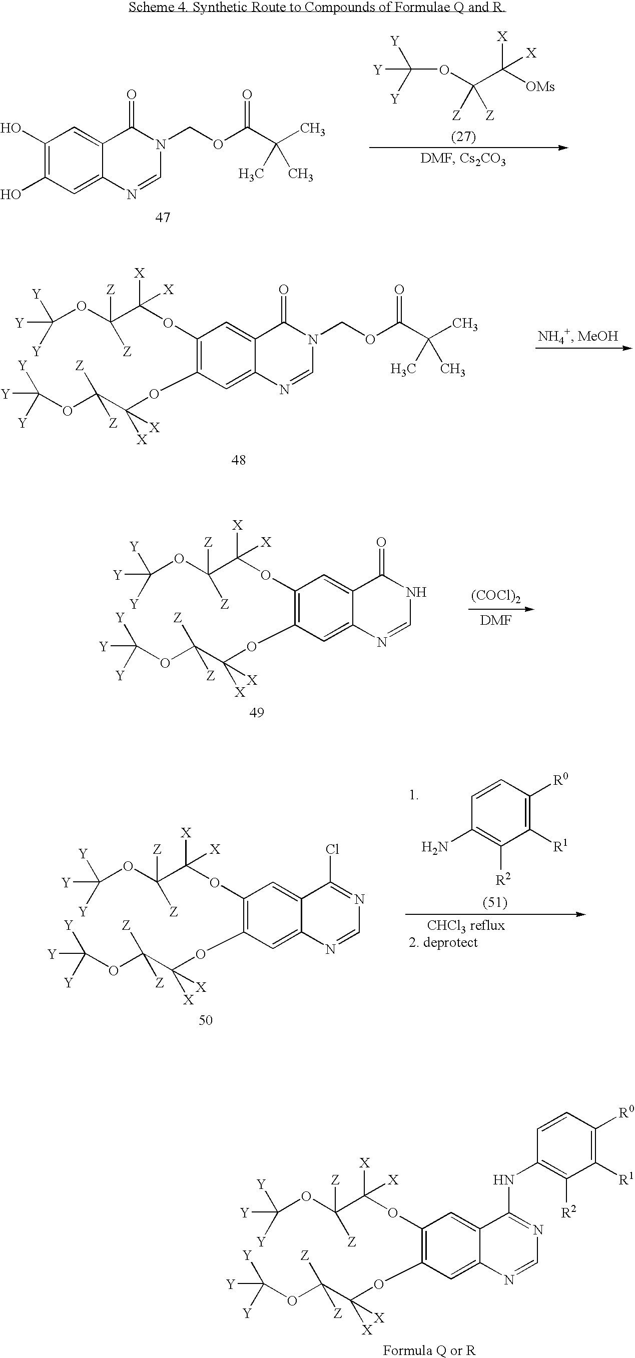

Scheme 4 depicts an alternate general synthetic scheme for the preparation of a compound of Formula Q or R, following the synthetic route described in Hennequin, L F et al., J. Med. Chem., 1999, 24: 5369-5389, for the preparation of compounds of similar structure. The starting dihydroxy compound 47, prepared as described by Hennequin et al., may be treated with an appropriately deuterated 2-methoxyethyl methanesulfonate 27 in cesium carbonate to form the protected 6,7-bis(2-methoxyethoxy)quinazolin-4(3H)-one 48. Intermediate 48 may be deprotected with ammonia in MeOH to form quinazoline 49, which subsequently may be converted to the corresponding chloroquinazoline 50 by treatment with oxalyl chloride in DMF. The chloroquinazoline 50 then may be coupled with aniline 51 to form a compound of Formula Q or R^{X} or an intermediate for synthesis thereof. Useful examples of intermediate 51 include the following anilines:

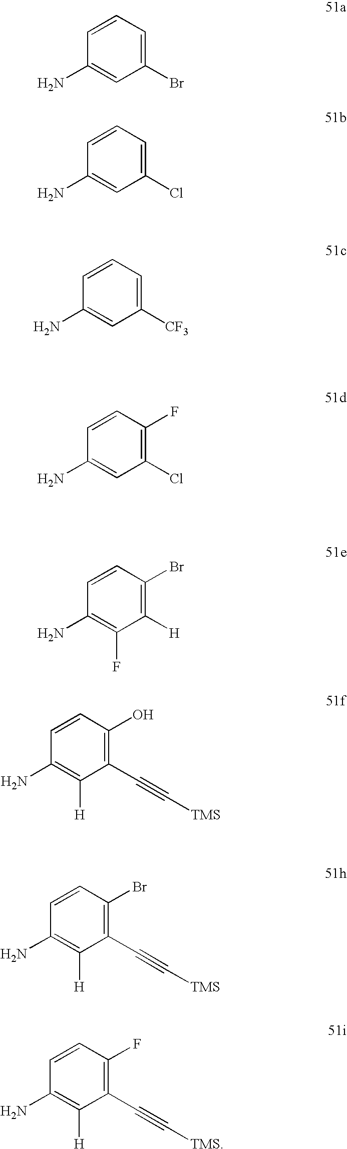

Anilines 51a-51e are commercially available. Anilines 51f and 51h may be synthesized as outlined in Schemes 5a and 5b below. Aniline 51i may be synthesized as described in J Org Chem, 1981, 2280-2296.

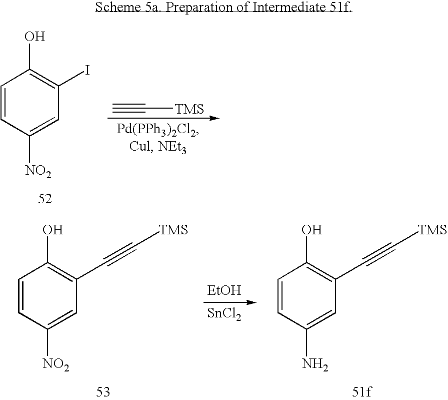

Scheme 5a depicts the synthesis of TMS-protected aniline 51f based on a procedure described in J Org Chem, 1981, 2280-2296. The starting material, 2-iodo-4-nitrophenol 52, prepared as described in J Org Chem, 2005, 70: 2445-2454, may be converted to 4-nitro-2-((trimethylsilyl)ethynyl)phenol 53 in the presence of ethynyltrimethylsilane, copper iodide, triethylamine and Pd(PPh₃)₂Cl₂. The protected nitrophenol 53 may be reduced by reaction with stannous chloride to produce the TMS-protected aniline 51f.

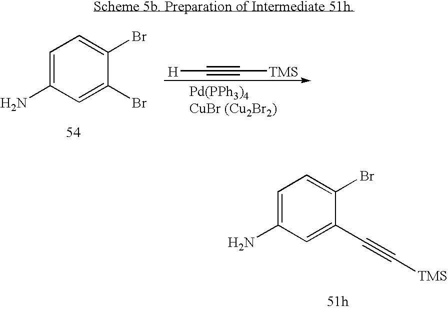

Intermediate 51h may be prepared as outlined in Scheme 5b, above, using conditions described in J Org Chem, 1989, 54: 4453-4457, for the preparation of similar compounds. Commercially available 3,4-dibromoaniline 54 may be coupled with ethynyltrimethylsilane under similar conditions to those described above (Scheme 5a) to produce the TMS-protected aniline 51h.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R⁰, W, X, Y, Z, etc.) or not. The suitability of a chemical group in a compound structure for use in synthesis of another compound structure is within the knowledge of one of ordinary skill in the art.

Additional methods of synthesizing compounds of the formulae herein and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides compositions comprising an effective amount of a compound of the formulae herein (e.g., Formula Q, R, A or I), or a pharmaceutically acceptable salt, of said compound; and an acceptable carrier. In one embodiment, the composition is a pyrogen-free composition. In another embodiment, a composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets and sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain preferred embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

A specialized formulation for compounds of the formulae herein (e.g., Formula Q, R, A or I) is a nanoparticulate formulation as disclosed for example in WO 2006110811.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardsley, N.Y., 1970, 537. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

The size of the dose required for the therapeutic or prophylactic treatment of a particular proliferative disease will necessarily be varied depending on the subject treated, the route of administration, and the severity of the illness being treated. Such dosages can be found in U.S. Pat. No. 5,770,599. The compounds of the invention will normally be administered to a subject at a unit dose within the range of about 5 mg to about 10,000 mg per square meter body area of the subject, i.e. from about 0.1 mg/kg to about 200 mg/kg, providing a therapeutically-effective dose. A unit dose form such as a tablet or capsule will usually contain, for example from about 1 mg to about 250 mg of active ingredient. Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of the present invention further comprises a second therapeutic agent. The second therapeutic agent includes any compound or therapeutic agent known to have or that demonstrates advantageous properties when administered with erlotinib. Such agents are described in detail in U.S. Pat. No. 5,770,599; WO 2001/076586; WO 2002/005791; WO 2001/070255; WO 2003/088971; WO 2004/014426; WO 2005/000213; WO 2005/004872; WO 2005/046665; WO 2005/052005; WO 2005117887; WO 2005/117888; WO 2005/117877; WO 2005/117915; WO 2005/117916; WO 2006/122227; WO 2006/026313; WO 2004/035057; WO 2006/099396; WO 2006/090930; WO 2006/047716; WO 2006/110175; WO 2006081985; WO 2006082428; WO 2007/106503; WO 2007056244; WO 2007054573; WO 2007075554; and WO 2007/127951; the disclosures of which are incorporated herein by reference.

Preferably, the second therapeutic agent is an agent useful in the treatment or prevention of a disease or condition selected from cancer, inflammation, angiogenesis, vascular restenosis, immunological disorder, pancreatitis, kidney disease, blastocyte maturation and implantation, psoriasis, or benign prostatic hypertrophy (BPH).

In one embodiment, the second therapeutic agent is selected from 2-deoxy-2-[18F]fluoro-D-glucose, 3′-deoxy-3′-[18F]fluorothymidine, 5-fluorouracil, AV412, avastin, bevacizumab, bexarotene, bortezomib, calcitriol, canertinib, capecitabine, carboplatin, celecoxib, cetuximab, CHR-2797, cisplatin, dasatinib, digoxin, enzastaurin, etoposide, everolimus, fulvestrant, gefitinib, gemcitabine, genistein, imatinib, irinotecan, lapatinib, lenalidomide, letrozole, leucovorin, matuzumab, oxaliplatin, paclitaxel, panitumumab, pegfilgrastim, pegylated alfa-interferon, pemetrexed, Polyphenon® E, satraplatin, sirolimus, sorafenib, sutent, sulindac, sunitinib, taxotere, temodar, temozolomide, temsirolimus, TGO1, tipifarnib, trastuzumab, valproic acid, vinflunine, volociximab, vorinostat, and XL647.

In a more specific embodiment, the second therapeutic agent is bevacizumab.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention is in the range of about 10 mg to about 2000 mg per treatment. In a more specific embodiment the amount is in the range of about 25 mg to about 750 mg, or from about 50 mg to about 300 mg, or most specifically from about 100 mg to about 150 mg per treatment. Treatment may be administered as an oral dose, an intravenous dose or a combination thereof. The present compounds may be administered once or twice daily, preferably once daily. Alternatively, treatment may be administered as a once-weekly bolus, for example as an oral dose of 100-2000 mg, or as an iv infusion of 1.5 mg/kg to 30 mg/kg.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for erlotinib.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

If the second therapeutic agents referenced above act synergistically with the compounds of this invention it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of inhibiting the activity of a Human Epidermal Growth Factor Receptor Type 1/Epidermal Growth Factor Receptor (HER1/EGFR) tyrosine kinase in a cell, comprising contacting a cell with one or more compounds of Formula Q, R, A or I herein.

According to another embodiment, the invention provides a method of treating a subject suffering from or susceptible to a disease that is beneficially treated by erlotinib comprising the step of administering to the subject in need thereof an effective amount of a compound or a composition of this invention. Such diseases are well known in the art and are disclosed, for example, in U.S. Pat. No. 5,770,599, U.S. Pat. No. 5,747,498, EP 1,110,953, EP 817,775, and U.S. Pat. No. 6,476,040. In particular, the invention provides a method of treating a subject suffering from or susceptible to cancer, inflammation, angiogenesis, vascular restenosis, immunological disorder, pancreatitis, kidney disease, blastocyte maturation and implantation, psoriasis, or benign prostatic hypertrophy (BPH).

In still another embodiment, the patient suffering from or susceptible to any of the aforementioned diseases or conditions is a smoker. In still another embodiment, the patient suffering from or susceptible to any of the aforementioned diseases or conditions is a non-smoker.

In one particular embodiment, the method of this invention is used to treat a patient suffering from or susceptible to a disease or condition selected from non-small cell lung cancer, ovarian cancer, colorectal cancer, head and neck cancer, brain cancer, bladder cancer, sarcoma, prostate cancer, melanoma, cervical cancer, solid tumors, astrocytoma, breast cancer, pancreatic cancer, glioblastoma multiform, renal cancer, digestive/gastrointestinal cancer, liver cancer, gynecological cancers, CNS tumors, thymoma, and gastric cancer.

In another particular embodiment, the method of this invention is used to treat a patient suffering from or susceptible to a disease or condition selected from non-small cell lung cancer and pancreatic cancer.

The compounds of the invention also have utility in the treatment of additional disorders of cellular growth in which aberrant cell signaling by way of receptor tyrosine kinase enzymes or non-receptor tyrosine kinase enzymes, including as yet unidentified tyrosine kinase enzymes, are involved. Such disorders include, for example, inflammation, angiogenesis, vascular restenosis, immunological disorder, pancreatitis, kidney disease and blastocyte maturation and implantation. Additionally, the compounds of the invention can be used to treat other diseases involving excessive cellular proliferation such as psoriasis and benign prostatic hypertrophy (BPH).

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with erlotinib. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include a method of treating a patient suffering from or susceptible to cancer comprising the step of co-administering a compound of Formula Q, A, I or R and a second therapeutic agent selected from 2-deoxy-2-[18F]fluoro-D-glucose, 3′-deoxy-3′-[18F]fluorothymidine, 5-fluorouracil, AV412, avastin, bevacizumab, bexarotene, bortezomib, calcitriol, canertinib, capecitabine, carboplatin, celecoxib, cetuximab, CHR-2797, cisplatin, dasatinib, digoxin, enzastaurin, etoposide, everolimus, fulvestrant, gefitinib, gemcitabine, genistein, imatinib, irinotecan, lapatinib, lenalidomide, letrozole, leucovorin, matuzumab, oxaliplatin, paclitaxel, panitumumab, pegfilgrastim, pegylated alfa-interferon, pemetrexed, Polyphenon® E, satraplatin, sirolimus, sorafenib, sutent, sulindac, sunitinib, taxotere, temodar, temozolomide, temsirolimus, TGO1, tipifarnib, trastuzumab, valproic acid, vinflunine, volociximab, vorinostat, and XL647 to the patient in need thereof.

In a more specific embodiment, the co-administered second therapeutic agent is bevacizumab.

In an even more specific embodiment, the co-administered second therapeutic agent is bevacizumab and the patient is suffering from non-small cell lung cancer.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In another aspect, the invention provides the use of a compound of the formulae herein, or a pharmaceutically-acceptable salt thereof, in the manufacture of a medicament for use in the production of an anti-proliferative effect in a subject.

In another embodiment, the invention provides a method of modulating the activity of cell surface tyrosine receptor kinases, including epidermal growth factor receptor kinases (EGFR), in a cell comprising contacting the cell with one or more compounds of any of the formulae herein.

In yet another aspect, the invention provides the use of a compound of the formulae herein (e.g., Formula Q, R, A or I) alone or together with one or more of the above-described second therapeutic agents in the manufacture of a medicament, either as a single composition or as separate dosage forms, for treatment or prevention in a subject of a disease, disorder or symptom set forth above. Another aspect of the invention is a compound of the formulae herein for use in the treatment or prevention in a subject of a disease, disorder or symptom thereof delineated herein.

### Pharmaceutical Kits

The present invention also provides kits for use to treat non-small cell lung cancer (NSCLC), pancreatic cancer, ovarian cancer, colorectal cancer, head and neck cancer, brain cancer, bladder cancer, sarcoma, prostate cancer, melanoma, cervical cancer, solid tumors, astrocytoma, breast cancer, glioblastoma multiform, renal cancer, digestive/gastrointestinal cancer, liver cancer, gynecological cancers, CNS tumors, thymoma and gastric cancer. These kits comprise (a) a pharmaceutical composition comprising a compound of Formula Q, R, A or I or a pharmaceutically acceptable salt thereof, wherein said pharmaceutical composition is in a container; and (b) instructions describing a method of using the pharmaceutical composition to treat non-small cell lung cancer (NSCLC), pancreatic cancer, ovarian cancer, colorectal cancer, head and neck cancer, brain cancer, bladder cancer, sarcoma, prostate cancer, melanoma, cervical cancer, solid tumors, astrocytoma, breast cancer, glioblastoma multiform, renal cancer, digestive/gastrointestinal cancer, liver cancer, gynecological cancers, CNS tumors, thymoma and gastric cancer.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### EXAMPLES

### Example 1

Synthesis of Various Deuterated Methoxyethyl Methane Sulfonate Intermediates 27. Intermediates 27 were prepared as outlined in Schemes 3a and 3b.

### A. (Methoxy-d₃)ethyl-d₄ Methanesulfonate (27-d7)

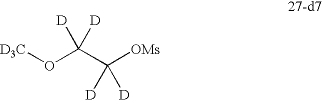

To a solution of 2-methoxyethanol-d₇ 35 [99 atom % D, CDN isotopes] (0.65 g, 7.72 mmol) in methylene chloride (5.00 mL) was added methanesulfonyl chloride 36 (1.00 mL, 11.59 mmol) and triethylamine (2.15 mL, 15.45 mmol) and the solution was stirred at room temperature for 1 hour. Water (10 mL) was added to the reaction mixture and the organic layer was separated, dried over Na₂SO₄ and concentrated in vacuo to give 27-d7 (0.90 g, 80%). ¹H NMR (400 MHz, CDCl₃): δ 3.10 (s, 3H).

### B. (Methoxy-d₃)-2,2-d₂-ethyl Methanesulfonate (27-d5)

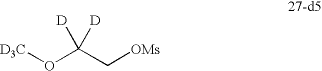

Step 1. (2-(Methoxy-d₃)-2,2-d₂-ethoxy)methyl)benzene (38-d5). To a solution of 2-benzyloxy-2,2-d₂-ethanol 37-d2 (5.00 g, 32.2 mmol, prepared as described by Bird, I et al., Journal of Labelled Compounds and Radiopharmaceuticals, 1989, 27(2): 199-216 using LiAlD_{4 [}96 atom % D, Aldrich]) in methylene chloride (50.0 mL) was added methanesulfonyl chloride 36 (4.00 mL, 48.3 mmol) and triethylamine (6.70 mL, 48.3 mmol) and the solution was stirred at room temperature for 1 hour. To the reaction mixture was added water (25 mL) and the organic layer was separated, washed with satd. brine solution (15 mL), dried over Na₂SO₄ and concentrated in vacuo. The resulting crude mesylate intermediate (6.00 g, 25.5 mmol, 80%) was dissolved in DMF (20.0 mL) and combined with CD₃O⁻Na⁺ 39-d3, which was produced in situ by incubating CD₃OD [99.8 atom % D, Aldrich] (2.00 mL, 51.0 mmol) in DMF (30.0 mL) with sodium hydride (2.00 g, 84.19 mmol, 55% dispersion in oil) and stirring at room temperature for 30 minutes (min). The mesylate/CD₃O⁻Na⁺ solution was stirred at room temperature for 6 hours. Water (25 mL) was added to the reaction mixture and the solution was extracted with methyl t-butyl ether (2×15 mL). The organic layer was dried over Na₂SO₄ and concentrated in vacuo to give 38-d5 (4.00 g, 85%).

Step 2. (Methoxy-d₃)-2,2-d₂-ethyl Methanesulfonate (27-d5). To a solution of (2-(methoxy-d₃)-2,2-d₂-ethoxy)methyl)benzene 38-d5 (4.00 g, 23.4 mmol) in THF (20 mL) was added 10% Pd/C (1.00 g) and the solution was subjected to hydrogenation for 6.0 hours. The resulting mixture was filtered through a pad of Celite. To the resulting filtrate was added triethylamine (5.22 mL, 37.5 mmol) and methanesulfonyl chloride 36 (3.25 mL, 37.5 mmol) and the solution was stirred at room temperature overnight. To the solution was added water (10 mL) and the reaction mixture was extracted with EtOAc (2×10 mL). The organic layer was dried over Na₂SO₄ and concentrated in vacuo to give 27-d5 (2.50 g, 68%). ¹H NMR (400 MHz, CDCl₃): δ 4.40 (s, 2H), 3.10 (s, 3H).

### C. Methoxy-2,2-d₂-ethyl Methanesulfonate (27-d2)

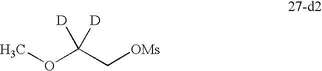

Step 1. (2-Methoxy-2,2-d₂-ethoxy)methyl)benzene (38-d2). To a solution of 2-benzyloxy-2,2-d₂-ethanol 37-d2 (4.50 g, 28.9 mmol, see B above) in methylene chloride (40.0 mL) was added methanesulfonyl chloride 36 (3.60 mL, 43.49 mmol) and triethylamine (6.00 mL, 43.5 mmol) and the solution was stirred at room temperature for 1 hour. Water (20 mL) was added to the reaction mixture and the organic layer was separated, washed with satd. brine solution (15 mL), dried over Na₂SO₄ and concentrated in vacuo. The resulting crude mesylate intermediate (6.00 g, 25.5 mmol, 80%) was dissolved in DMF (20.0 mL) and combined with CH₃O⁻Na⁺ 39, which was produced in situ by incubating CH₃OD [99.8 atom % D, Aldrich] (2.00 mL, 55.9 mmol) in DMF (30.0 mL) with sodium hydride (2.20 g, 92.3 mmol, 60% dispersion in oil) and stirring at rt for 30 min. The mesylate/CH₃O⁻Na⁺ solution was stirred at room temperature for 6 hours. Water (25 mL) was added to the reaction mixture and the solution was extracted with methyl t-butyl ether (2×15 mL). The combined organic extracts were dried over Na₂SO₄ and concentrated in vacuo to give 38-d2 (4.00 g, 85%).

Step 2. Methoxy-2,2-d₂-ethyl Methanesulfonate (27-d2). To a solution of (2-methoxy-2,2-d₂-ethoxy)methyl)benzene 38-d2 (4.00 g, 23.08 mmol) in THF (70 mL) was added 10% Pd/C (1.00 g) and the solution was subjected to hydrogenation for 6.0 hours. The resultant mixture was filtered through a pad of Celite and to the filtrate was added triethylamine (5.00 mL, 35.7 mmol) followed by methanesulfonyl chloride 36 (2.30 mL, 35.7 mmol). The resultant solution was stirred at rt overnight. To the solution was added water (10 mL) and the reaction mixture was extracted with EtOAc (2×10 mL). The combined organic extracts were dried over Na₂SO₄ and concentrated in vacuo to give 27-d2 (2.50 g, 68%). ¹H NMR (400 MHz, CDCl₃): δ 4.40 (s, 2H), 3.70 (s, 2H), 3.10 (s, 3H).

### D. (Methoxy-d₃)ethyl Methanesulfonate (27-d3)

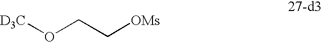

Step 1. (2-(Methoxy-d₃)ethoxy)methyl)benzene (38-d3). To a solution of 2-benzyloxyethanol 37 (8.10 g, 53.2 mmol) in methylene chloride (80.0 mL) was added methanesulfonyl chloride 36 (6.60 mL, 79.7 mmol) and triethylamine (11.1 mL, 79.78 mmol) and the solution was stirred at room temperature for 1 hour. Water (25 mL) was added to the reaction mixture and the organic layer was separated, washed with satd. brine solution (15 mL), dried over Na₂SO₄ and concentrated in vacuo. The crude mesylate intermediate (3.73 mL, 92.0 mmol) was dissolved in DMF (100.0 mL) and combined with CD₃O^{-Na}⁺ 39-d3 which was produced in situ by incubating CD₃OD [99.8 atom % D, Aldrich] (3.73 mL, 92.0 mmol) in DMF (100.0 mL) with sodium hydride (3.70 g, 151.8 mmol, 55% dispersion in oil) and stirring at room temperature for 30 min. The mesylate/CD₃O⁻Na⁺ solution was stirred at room temperature for 6 hours. Water (25 mL) was added to the reaction mixture and the solution was extracted with methyl t-butyl ether (2×15 mL). The combined organic extracts were dried over Na₂SO₄ and concentrated in vacuo to give 38-d3 (7.00 g, 95%).

Step 2. (Methoxy-d₃)ethyl Methanesulfonate (27-d3). To a solution of (2-(methoxy-d₃)ethoxy)methyl)benzene 38-d3 (2.70 g, 15.9 mmol) in THF (20 mL) was added 10% Pd/C (1.00 g, 50% wet) and the solution was subjected to hydrogenation for 6.0 hours. The resultant reaction mixture was filtered through a pad of Celite. To the filtrate was added triethylamine (2.90 mL, 21.1 mmol) followed by methanesulfonyl chloride 36 (1.70 mL, 21.1 mmol) and the solution was stirred at room temperature overnight. To the resulting solution was added water (10 mL) and the mixture was extracted with EtOAc (2×10 mL). The combined organic extracts were dried over Na₂SO₄ and concentrated in vacuo to give 27-d3 (1.70 g, 77%). MS (M+H): 158.

### Example 2

Synthesis of 4-Amino-2-((trimethylsilyl)ethynyl)phenol (51f). Intermediate 51f was prepared as outlined in Scheme 5a.

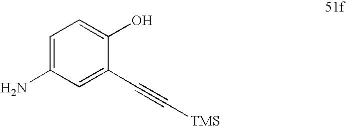

Step 1. 4-Nitro-2-((trimethylsilyl)ethynyl)phenol (53). To a solution of 2-iodo-4-nitrophenol 52 (2.00 g, 7.54 mmol, prepared as described in J. Org. Chem. 2005, 70, 2445-2454) in dioxane (15 mL) was added Pd(Ph₃)₂Cl₂ (0.400 g), CuI (0.200 g), and triethylamine (2.40 mL). The resulting mixture was stirred at room temperature for 2 hours. The mixture was then filtered through Celite and the solvent evaporated in vacuo to give the crude product. Purification by column chromatography afforded 0.500 g of 53 (29%). ¹H NMR (400 MHz, CDCl₃): δ 0.04 (s, 9H), 7.0 (d, J=8.8.Hz, 1H), 8.15 (dd, J=8.8 Hz, 2.8 Hz), 8.28 (d, 1H, J=2.8 Hz).

Step 2. 4-Amino-2-((trimethylsilyl)ethynyl)phenol (51f). To a stirred solution of 53 (3.60 g, 15.3 mmol) in EtOH (80 mL) was added SnCl₂.2H₂O (17.2 g, 76.4 mmol). The mixture was stirred at reflux for 2 hours, then was concentrated in vacuo. The residue was basified to pH=8, then was taken up in EtOAc (2×5 mL) and evaporated to give 51f (2.00 g, 64%). ¹H NMR (400 MHz, CDCl₃): δ 0.3 (s, 9H), 6.62 (dd, 1H, J=8.8, 2.8 Hz), 6.67 (d, 1H, J=2.8 Hz), 6.77 (d, 1H, J=8.8 Hz). MS (M+H): 206.

### Example 3

Synthesis of 4-(6,7-Bis(2-(methoxy-d₃)-2,2-d₂-ethoxy)quinazolin-4-ylamino)-2-ethynylphenol (Compound 138). Compound 138 was prepared as outlined in Scheme 4.

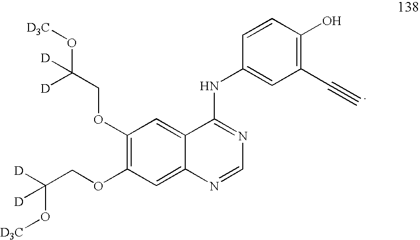

Step 1. (6,7-Bis(2-(methoxy-d₃)-2,2-d₂-ethoxy)-4-oxoquinazolin-3(4H)-yl)methyl pivalate (48-d10). To a solution of the dihydroxy compound 47 (0.500 g, 1.71 mmol, prepared as described in Hennequin, L F et al., J. Med. Chem., 1999, 24: 5369-5389) in DMF (5.0 mL) was added Cs₂CO₃ (0.650 g, 4.10 mmol) and 2-(methoxy-d₃)-2,2-d₂-ethyl methanesulfonate 27-d5 (0.806 g, 5.13 mmol). The solution was stirred at 60° C. for 3 hours. DMF was removed from the reaction mixture under reduced pressure and water (5 mL) was added. The solution was extracted with EtOAc (2×5 mL). The combined organic extracts were dried over Na₂SO₄ and concentrated in vacuo to give crude 48-d10 (0.200 g, 40%).

Step 2. 6,7-Bis(2-(methoxy-d₃)-2,2-d₂-ethoxy)quinazolin-4(3H)-one (49-d10). To the dialkylated product 48-d10 (0.150 g, 0.49 mmol) was added ammonia in MeOH (4.0 mL). The reaction mixture was stirred at room temperature overnight then was concentrated in vacuo and diethyl ether was added to the residue. The solids formed were filtered and dried under vacuum to afford 49-d10 (0.100 g, 71%).

Step 3. 4-Chloro-6,7-bis(2-(methoxy-d₃)-2,2-d₂-ethoxy)quinazoline (50-d10). To a solution of 49-d10 (0.100 g, 0.327 mmol) in CHCl₃ (2.0 mL) was added DMF (cat. amount) and oxalyl chloride (0.1 mL, 0.443 mmol). The solution was stirred at 0° C. for 15 min then was heated to reflux and stirred for 6 hours. To the reaction mixture was added satd. NaHCO₃ and the organic layer was separated. The organic layer containing 50-d10 was directly taken to the next step.

Step 4. 4-(6,7-Bis(2-methoxyethoxy)quinazolin-4-ylamino)-2-ethynylphenol (Compound 138). To a solution of 50-d10 (0.05 g, 0.155 mmol) in CHCl₃ (2.0 mL) was added TMS-protected aniline 51f (0.034 g, 0.170 mmol) dissolved in CHCl₃ (1 mL) and the solution was stirred at reflux temperature for 4 hours. The reaction mixture was concentrated in vacuo and the solid obtained was washed with diethyl ether. To the crude product (0.05 g, 0.101 mmol) was added with K₂CO₃ (0.041 g, 0.303 mmol) in MeOH (2 mL) and the solution was stirred for 4 hours at room temperature. The reaction mixture was concentrated in vacuo and the residue was purified by column chromatography with neutral alumina to give Compound 138 (7 mg). ¹H NMR (400 MHz, DMSO-d₆) δ: 4.10 (s, 1H), 4.24 (s, 4H), 6.88-6.90 (m, 1H), 7.17 (s, 1H), 7.54-7.55 (m, 1H), 7.81 (s, 1H), 7.62-7.65 (m, 1H), 7.81 (s, 1H), 8.38 (s, 1H), 9.35 (s, 1H), 9.87 (s, 1H). MS (M+H): 420.

### Example 4

Synthesis of N-(3-Ethynyl-4-fluorophenyl)-6,7-bis(2-(methoxy-d₃)-2,2-d₂-ethoxy)quinazolin-4-amine (Compound 135). Compound 135 was prepared as outlined in Scheme 4.

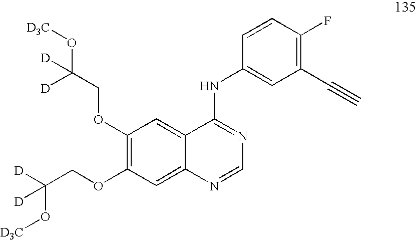

N-(3-Ethynyl-4-fluorophenyl)-6,7-bis(2-(methoxy-d₃)-2,2-d₂-ethoxy)quinazolin-4-amine (Compound 135). To a solution of 50-d10 (0.200 g, 0.62 mmol, see Example 3) in CHCl₃ (5.0 mL) was added aniline 51i (0.139 g, 0.682 mmol, prepared as described in J. Org. Chem., 1981, 2280-2296) dissolved in CHCl₃ (1.0 mL) and the solution was stirred at reflux for 4 hours. The reaction mixture was concentrated in vacuo and the solid obtained was washed with diethyl ether to give the product (0.200 g, 60%). To the crude product (0.200 g, 0.4 mmol) was added ammonia in MeOH (4 mL) and the solution was stirred for 4 hours at room temperature. The reaction mixture was concentrated in vacuo and the residue was purified by column chromatography to give Compound 135 (40 mg). ¹H NMR (400 MHz, DMSO-d₆): δ 4.26 (s, 4H), 4.50 (s, 1H), 6.50 (s, 1H), 7.21 (s, 1H), 7.30-7.34 (m, 1H), 7.84-7.88 (m, 2H), 7.99-8.00 (m, 1H), 8.46 (s, 1H), 9.48 (s, 1H). MS (M+H): 422.

### Example 5

Synthesis of 4-(6,7-Bis(2-(methoxy-d₃)ethoxy-d₄)quinazolin-4-ylamino)-2-ethynylphenol (Compound 124). Compound 124 was prepared as outlined in Scheme 4.

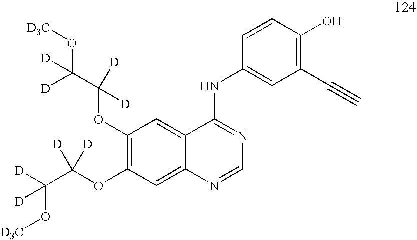

Step 1. (6,7-Bis(2-(methoxy-d₃)ethoxy-d₄)-4-oxoquinazolin-3(4H)-yl)methyl pivalate (48-d14). To a solution of the dihydroxy compound 47 (0.500 g, 1.71 mmol, see Example 3) in DMF (5.0 mL) was added Cs₂CO₃ (0.650 g, 4.10 mmol) and 2-methoxyethyl methanesulfonate-d7 27-d7 (0.806 g, 5.13 mmol). The mixture was stirred at 60° C. for 3 hours. DMF was removed in vacuo and water (5 mL) was added to the residue. The mixture was extracted with EtOAc (2×5 mL). The combined EtOAc layers were dried over Na₂SO₄ and concentrated in vacuo to give 48-d14 (0.200 g, 40%).

Step 2. 6,7-Bis(2-(methoxy-d₃)ethoxy-d₄)quinazolin-4(3H)-one (49-d14). To the dialkylated product 48-d14 (0.200 g, 0.49 mmol) was added ammonia in MeOH (4.0 mL) and the reaction mixture was stirred at room temperature overnight. The reaction mixture was concentrated in vacuo and diethyl ether was added to the residue. The solids formed were filtered and dried under vacuum to afford 49-d14 (0.100 g, 70%).

Step 3. 4-Chloro-6,7-bis(2-(methoxy-d₃)ethoxy-d₄)quinazoline (50-d14). To a solution of 49-d14 (0.100 g, 0.327 mmol) in CHCl₃ (2.0 mL) was added DMF (cat. amount) and oxalyl chloride (0.1 mL, 0.443 mmol) and the solution was stirred at 0° C. for 15 min. The reaction mixture was heated to reflux for 6 hours. To the resulting reaction mixture was added satd. NaHCO₃. The organic layer was separated and directly taken to the next step.

Step 4. 4-(6,7-Bis(2-(methoxy-d₃)ethoxy-d₄)quinazolin-4-ylamino)-2-((trimethylsilyl)ethynyl)phenol. To a solution of 50-d14 (0.100 g, 0.306 mmol) in CHCl₃ (2 mL) was added the TMS-protected aniline 51f (0.069 g, 0.337 mmol) dissolved in isopropanol (1 mL) and the solution was stirred at reflux for 4 hours. The reaction mixture was concentrated in vacuo and the solid obtained was washed with diethyl ether. The desired product was isolated using preparative TLC to give 8 mg. ¹H NMR (400 MHz, DMSO-d₆): δ 3.90 (s, 1H), 6.88-6.90 (m, 1H), 7.17 (s, 1H), 7.54-7.55 (m, 1H), 7.81 (s, 1H), 7.62-7.65 (m, 1H), 7.80 (s, 1H), 8.39 (s, 1H), 9.30 (s, 1H), 9.84 (s, 1H). MS (M+H): 496.

Step 5. 4-(6,7-Bis(2-(methoxy-d₃)ethoxy-d₄)quinazolin-4-ylamino)-2-ethynylphenol (Compound 124). A mixture of the TMS-protected phenol produced in the prior step and

K₂CO₃ in MeOH was stirred for 4 hours at room temperature. The reaction mixture was concentrated in vacuo and the residue purified by column chromatography with neutral alumina to give the product Compound 124. Due to the unstable nature of Compound 124, no analytical characterization was performed.

### Example 6

Synthesis of N-(3-Ethynyl-4-fluorophenyl)-6,7-bis(2-(methoxy-d₃)ethoxy-d₄)quinazolin-4-amine (Compound 121). Compound 121 was prepared as outlined in Scheme 1.

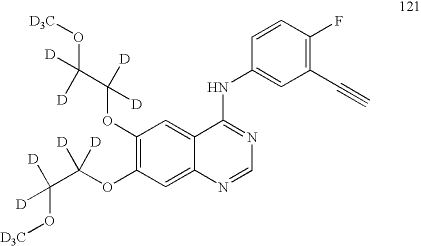

Step 1. 6,7-Dihydroxy-4(3H)-quinazolinone (11). A solution of 6,7-dimethoxy-4(3H)-quinazolinone 10 (3.0 g, 14.5 mmol) and 48% HBr (36 mL) was heated to reflux at 100° C. for 12 hours. The reaction mixture was cooled to room temperature and the solids were removed by filtration then neutralized with aqueous NH₃ (pH=8). The resulting solution was filtered and the solids were washed with water and dried to give 6,7-dihydroxy-4(3H)-quinazolinone 11 as an off-white crystalline solid (2.20 g, 84%).

Step 2. 6,7-Diacetoxy-4(3H)-quinazolinone (12). To 6,7-dihydroxy-4(3H)-quinazolinone 11 (2.20 g, 12.2 mmol) was added Ac₂O (13.3 ml) and a drop of pyridine and the resulting reaction mixture was heated to reflux at 120° C. for 2 hours. The reaction mixture was cooled to room temperature and the solvent was removed under reduced pressure. The residue was taken up in water and the mixture stirred for 1 hour at room temperature. The resulting precipitate was filtered and dried to give 6,7-diacetoxy-4(3H)-quinazolinone 12 as an off-white crystalline solid (1.70 g, 53%).

Step 3. 4-Chloroquinazoline-6,7-diyl diacetate (13). To a solution of 6,7-diacetoxy-4(3H)-quinazolinone 12 (3.20 g, 11.42 mmol) in CHCl₃ (60 ml) was added oxalyl chloride (2.2 ml, 17.3 mmol) dropwise at 0° C. The resulting reaction mixture was stirred at room temperature for 15 min then was gradually heated to reflux for 5 hours. The reaction mixture was cooled to 10° C. and the solution was quenched with aqueous sodium bicarbonate. The organic layer was separated and washed with brine then dried over Na₂SO₄ and directly taken to the next step without concentration.

Step 4. 4-(3-Ethynyl-4-fluorophenylamino)quinazoline-6,7-diol (26). To a solution of 13 (0.800 g, 2.10 mmol) in CHCl₃ (10 mL) was added 3-ethynyl-4-fluoroaniline 24d (0.311 g, 2.31 mmol, prepared as described in J Org Chem, 1981, 2280-2296) and the solution was stirred at reflux overnight. The solids obtained were filtered and dried to give the diacetyl product 25 (0.700 g, 70%). The product 25 was stirred with ammonia in MeOH (2 mL) for 1 hour at room temperature. The mixture was concentrated in vacuo and the residue was washed with water (5 mL) and filtered to give the product 26 (0.300 g, 55%).

Step 5. N-(3-Ethynyl-4-fluorophenyl)-6,7-bis(2-(methoxy-d₃)ethoxy-d₄)quinazolin-4-amine (Compound 121). To a solution of 26 (0.500 g, 1.69 mmol) in DMF (15 mL) was added K₂CO₃ (0.930 g, 6.70 mmol) and 2-(methoxy-d₃)ethyl-d₄ methanesulfonate 27-d7 (0.59 g, 3.71 mmol). The solution was stirred at 90° C. for 4 hours. DMF was removed in vacuo and the residue was purified by column chromatography to give 22 mg of Compound 121. ¹H NMR (400 MHz, DMSO-d₆): δ 4.50 (s, 1H), 7.22 (s, 1H), 7.31-7.36 (m, 1H), 7.83 (s, 1H), 7.86-7.89 (m, 1H), 8.00-8.47 (m, 1H), 8.47 (s, 1H), 9.49 (s, 1H). MS (M+H): 426.

### Example 7

Synthesis of N-(3-Ethynylphenyl)-6,7-bis(2-(methoxy-d₃)ethoxy-d₄)quinazolin-4-amine (Compound 120). Compound 120 was prepared as outlined in Scheme 1.

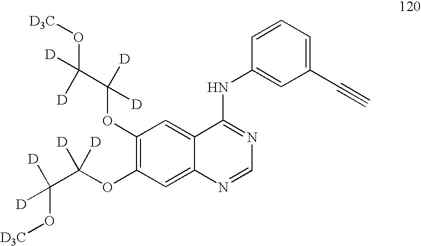

Step 1. N-(3-Ethynylphenyl)-6,7-diacetoxy-4-quinazolinamine hydrochloride (25). To the solution of 4-chloroquinazoline-6,7-diyl diacetate 13 in CHCl₃ produced in Example 6 was added ethynyl aniline 24e (1.19 ml, 11.42 mmol, commercially available). The reaction mixture was heated to reflux overnight, was cooled to room temperature and was filtered to give 25 as a solid (3.0 g, 94%).

Step 2. N-(3-Ethynylphenyl)-6,7-dihydroxy-4-quinazolinamine hydrochloride (26). To the hydrochloride salt 25 (4.2 g, 11.62 mmol) in methanol (30 ml) was added 25% aqueous ammonia (4.73 ml) and the solution was stirred for 4 hours. The reaction mixture was filtered and the solid was washed with water to give 26 (3.0 g, 93%) as a brownish yellow solid.

Step 3. N-(3-Ethynylphenyl)-6,7-bis(2-(methoxy-d₃)ethoxy-d₄)quinazolin-4-amine (Compound 120). To a solution of the dihydroxy compound 26 (0.800 g, 2.88 mmol) in DMF (25 mL) was added K₂CO₃ (1.59 g, 11.52 mmol) and 2-(methoxy-d₃)ethyl-d methanesulfonate 27-d7 (1.12 g, 7.21 mmol). The solution was stirred at room temperature for 15 min and then heated to 60° C. and stirred for 2 hours. The resulting mixture was stripped of DMF in vacuo and the residue was taken up in water (10 mL). The aqueous solution was extracted with EtOAc (2×5 mL) and the combined organic extracts were dried over Na₂SO₄ and concentrated in vacuo. The residue was taken up in diethyl ether (3 mL) and the solids were filtered to give Compound 120 (0.500 g, 43%). ¹H NMR (400 MHz, DMSO-d₆): δ 4.10 (s, 1H), 7.10 (m, 1H), 7.30 (m, 1H), 7.80 (m, 1H), 7.90 (s, 1H), 8.50 (s, 1H), 9.50 (s, 1H). MS (M+H): 408.

### Example 8

### Synthesis of N-(3-Bromophenyl)-6,7-bis(2-(methoxy-d₃)-2,2-d₂-ethoxy)quinazolin-4-amine (Compound 105)

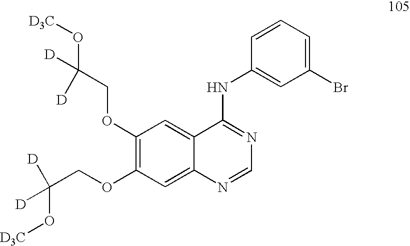

N-(3-Bromo)-6,7-bis(2-(methoxy-d₃)-2,2-d₂-ethoxy)quinazolin-4-amine (Compound 105). To a solution of 50-d10 (0.056 g, 0.174 mmol, see Example 3 for preparation) in CHCl₃ (2.0 mL) was added 3-bromoaniline 51a (21 μL, 0.191 mmol). The resulting solution was stirred at reflux for 15 hours and was then diluted with satd NaHCO₃ and extracted with EtOAc. The combined organic layers were dried (MgSO₄), filtered and concentrated in vacuo. The resulting residue was then purified by column chromatography (SiO₂, O-3% MeOH/CH₂Cl₂) to afford Compound 105 (20.4 mg, 26%). ¹H NMR (400 MHz, CDCl₃): δ 8.66 (s, 1H), 8.00-7.97 (m, 1H), 7.66-7.60 (M, 1H), 7.47 (s, 1H), 7.66-7.22 (m, 2H), 7.19 (s, 1H), 7.17 (s, 1H), 4.23 (s, 2H), 4.19 (s, 2H). MS (M+H): 476.

### Example 9

### Synthesis of N-(3-Chlorophenyl)-6,7-bis(2-(methoxy-d₃)-2,2-d₂-ethoxy)quinazolin-4-amine (Compound 106)

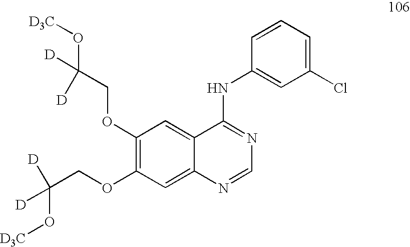

N-(3-Chloro)-6,7-bis(2-(methoxy-d₃)-2,2-d₂-ethoxy)quinazolin-4-amine (Compound 106). To a solution of 50-d10 (0.058 g, 0.180 mmol, see Example 3 for preparation) in CHCl₃ (2.0 mL) was added 3-chloroaniline 51b (21 μL, 0.198 mmol). The resulting mixture was stirred at reflux for 15 hours and was then diluted with satd NaHCO₃ and extracted with EtOAc. The combined organic layers were dried (MgSO₄), filtered and concentrated in vacuo. The resulting residue was purified by column chromatography (SiO₂, O-3% MeOH/CH₂Cl₂) to afford Compound 106 (37 mg, 49%). ¹H NMR (400 MHz, CDCl₃): δ 8.67 (s, 1H), 7.89 (t, J=2.0 Hz, 1H), 7.56 (ddd, J=0.8, 2.0, 8.1 Hz, 1H), 7.38 (br s, 1H), 7.31 (t, J=8.1 Hz, 1H), 7.19 (d, 1.8 Hz, 2H), 7.11 (ddd, J=1.0, 2.0, 8.1 Hz, 1H), 4.25 (s, 2H), 4.21 (s, 2H). MS (M+H): 414.

### Example 10

### Synthesis of N-(3-Trifluoromethylphenyl)-6,7-bis(2-(methoxy-d₃)-2,2-d₂-ethoxy)quinazolin-4-amine (Compound 108)

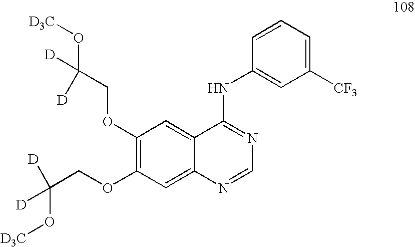

N-(3-Trifluoromethyl)-6,7-bis(2-(methoxy-d₃)-2,2-d₂-ethoxy)quinazolin-4-amine (108). To a solution of 50-d10 (0.058 g, 0.180 mmol, see Example 3 for preparation) in CHCl₃ (2.0 mL) was added 3-trifluoromethylaniline 51c (25 μL, 0.198 mmol) and the resulting solution was stirred at reflux for 15 hours. The reaction mixture was then diluted with satd NaHCO₃ and extracted with EtOAc. The combined organic layers were dried (MgSO₄), filtered and concentrated in vacuo. The resulting residue was purified by column chromatography (SiO₂, O-3% MeOH/CH₂Cl₂) to afford Compound 108 (33.7 mg, 42%). ¹H NMR (400 MHz, CDCl₃): δ 8.67 (s, 1H), 8.03-7.96 (m, 2H), 7.55-7.46 (m, 2H), 7.41-7.36 (m, 1H), 7.20 (s, 1H), 7.19 (s, 1H), 4.27 (s, 2H), 4.21 (s, 2H). MS (M+H): 448.

### Example 11

### Synthesis of N-(3-Chloro-4-fluorophenyl)-6,7-bis(2-(methoxy-d₃)-2,2-d₂-ethoxy)quinazolin-4-amine (Compound 115)

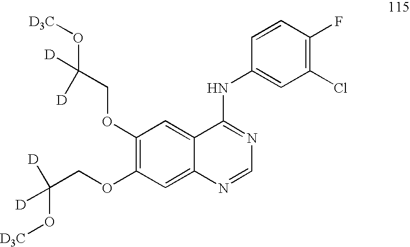

N-(3-Chloro-4-fluoro)-6,7-bis(2-(methoxy-d₃)-2,2-d₂-ethoxy)quinazolin-4-amine (Compound 115). To a solution of 50-d10 (0.058 g, 0.180 mmol, see Example 3 for preparation) in CHCl₃ (2.0 mL) was added 3-chloro-4-fluoroaniline 51d (29 mg, 0.198 mmol). The mixture was stirred at reflux for 15 hours and was then diluted with satd NaHCO₃ and extracted with EtOAc. The combined organic layers were dried (MgSO₄), filtered and concentrated in vacuo and the resulting residue was purified by column chromatography (SiO₂, 0-3% MeOH/CH₂Cl₂) to afford Compound 115 (42.5 mg, 55%). ¹H NMR (400 MHz, CDCl₃): δ 8.60 (s, 1H), 7.86 (dd, J=2.8, 6.6 Hz, 1H), 7.72 (br s, 1H), 7.56-7.49 (m, 1H), 7.22 (s, 1H), 7.13 (t, J=8.6 Hz, 1H), 7.12 (s, 1H), 4.20 (s, 2H), 4.16 (s, 2H). MS (M+H): 432.

### Example 12

### Synthesis of N-(4-Bromo-2-fluorophenyl)-6,7-bis(2-(methoxy-d₃)-2,2-d₂-ethoxy)quinazolin-4-amine (Compound 119)

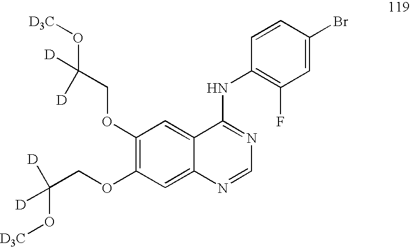

N-(4-Bromo-2-fluoro)-6,7-bis(2-(methoxy-d₃)-2,2-d₂-ethoxy)quinazolin-4-amine (Compound 119). To a solution of 50-d10 (0.058 g, 0.180 mmol, see Example 3 for preparation) in CHCl₃ (2.0 mL) was added 4-bromo-2-fluoroaniline (38 mg, 0.198 mmol). The resulting mixture was stirred at reflux for 15 hours and was then diluted with satd NaHCO₃ and extracted with EtOAc. The combined organic layers were dried (MgSO₄), filtered and concentrated in vacuo and the resulting residue was purified by column chromatography (SiO₂, O-3% MeOH/CH₂Cl₂) to afford Compound 119 (44.7 mg, 52%). ¹H NMR (400 MHz, CDCl₃): δ 8.68 (s, 1H), 8.53 (t, J=8.8 Hz, 1H), 7.37-7.29 (m, 3H), 7.25 (s, 1H), 7.21 (s, 1H), 4.30 (s, 2H), 4.28 (s, 2H). MS (M+H): 476.

### Example 13

Synthesis of 4-Bromo-3-ethynylaniline (24f). Intermediate 24f was prepared as generally outlined in Scheme 5b.

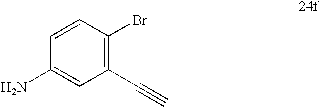

4-Bromo-3-ethynyl aniline (24f). To a solution of 3,4-dibromoaniline 54 (2.00 g, 7.97 mmol) and trimethylsilyl acetylene (1.10 mL, 7.97 mmol) in triethylamine (32 mL) was added copper(I) bromide (69.0 mg, 0.478 mmol) followed by palladium tetrakistriphenylphosphine (184 mg, 0.159 mmol). The reaction was stirred at reflux for 20 hours then cooled to room temperature. The reaction was then concentrated to remove triethylamine and the resulting residue was diluted with satd NaHCO₃ and extracted with EtOAc. The combined organic layers were dried (Na₂SO₄), filtered and concentrated in vacuo. The resulting residue was dissolved in methanol (80 mL) and K₂CO₃ (5.51 g, 39.9 mmol) was added. This mixture was stirred at room temperature for 15 hours then was diluted with water and the methanol was removed in vacuo. The resulting aqueous solution was then extracted with EtOAc and the combined organic layers were dried (Na₂SO₄), filtered, and concentrated in vacuo. The resulting residue was purified by column chromatography (SiO₂, O-15% EtOAc/Heptanes) to afford 24f (70 mg, 45%). ¹H NMR (400 MHz, CDCl₃): δ 7.30 (d, J=8.6 Hz, 1H), 6.84 (d, J=3.0 Hz, 1H), 6.54 (dd, J=2.8, 8.6 Hz, 1H), 3.31 (s, 1H). MS (M+H): 197.

### Example 14

### Synthesis of N-(4-Bromo-3-ethynyl phenyl)-6,7-bis(2-(methoxy-d₃)-2,2-d₂-ethoxy)quinazolin-4-amine (Compound 113)

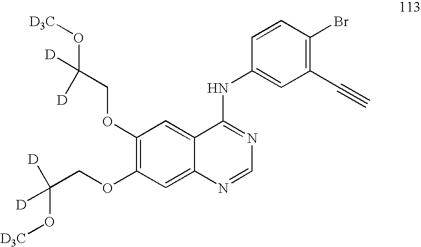

N-(4-Bromo-3-ethynyl)-6,7-bis(2-(methoxy-d₃)-2,2-d₂-ethoxy)quinazolin-4-amine (Compound 113). To a solution of 50-d10 (0.058 g, 0.180 mmol, see Example 3 for preparation) in CHCl₃ (2.0 mL) was added 4-bromo-3-ethynylaniline (24f) (39 mg, 0.198 mmol). The resulting solution was stirred at reflux for 15 h and was then diluted with satd NaHCO₃ and extracted with EtOAc. The combined organic layers were dried (MgSO₄), filtered and concentrated in vacuo. The resulting residue was purified by column chromatography (SiO₂, 0-3% MeOH/CH₂Cl₂) to afford Compound 113(15.9 mg, 18%). ¹H NMR (400 MHz, CDCl₃): δ 8.64 (s, 1H), 7.92 (d, J=2.5 Hz, 1H), 7.66 (dd, J=2.5, 8.8 Hz, 1H), 7.57 (d, J=8.6 Hz, 1H), 7.40 (br s, 1H), 7.17 (d, J=2.0 Hz, 1H), 4.24 (s, 2H), 4.19 (s, 2H), 3.39 (s, 1H). MS (M+H): 482.

### Example 15

Evaluation of Compound Stability in Human Liver Microsomes. Comparison of Compounds 120, 121, 135, 138 and Erlotinib.

The metabolic stability of compounds of the invention was tested using pooled liver microsomal incubations. Samples of the test compounds, exposed to pooled human liver microsomes, were analyzed using HPLC-MS (or MS/MS) detection. For determining metabolic stability, multiple reaction monitoring (MRM) was used to measure the disappearance of the test compounds.

Experimental Procedures. Human liver microsomes (“HLM”; 20 mg/mL) were obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) were purchased from Sigma-Aldrich.

Stock solutions of test compounds (7.5 mM) were prepared in DMSO. The 7.5 mM stock solutions were diluted to 50 μM in acetonitrile (ACN). The 20 mg/mL human liver microsomes were diluted to 0.625 mg/mL in 0.1 M potassium phosphate buffer, pH 7.4, containing 3 mM MgCl₂. The diluted microsomes (375 μL) were added to wells of a 96-well deep-well polypropylene plate in triplicate. A 10 μL aliquot of the 50 μM test compound solution was added to the microsomes and the mixture was pre-warmed for 10 minutes. Reactions were initiated by addition of 125 μL of pre-warmed NADPH solution (8 mM NADPH in 0.1M potassium phosphate buffer, pH 7.4, containing 3 mM MgCl₂). The final reaction volume was 0.5 mL and contained 0.5 mg/mL human liver microsomes, 1 μM test compound, and 2 mM NADPH in 0.1 M potassium phosphate buffer, pH 7.4, and 3 mM MgCl₂. The reaction mixtures were incubated at 37° C., and 50 μL aliquots were removed at 0, 5, 10, 20, and 30 minutes and added to shallow-well 96-well plates which contained 50 μL of ice-cold ACN with internal standard to stop the reactions. The plates were stored at 4° C. for 20 minutes after which 100 μL of water was added to the wells of the plate before centrifugation to pellet precipitated proteins. Supernatants were transferred to another 96-well plate and analyzed for amounts of parent remaining by LC-MS/MS using an Applied Bio-systems API 4000 mass spectrometer. 7-ethoxycoumarin was used as a positive control.

The in vitro t_{1/2} s for test compounds were calculated from the slopes of the linear regression of % parent remaining (ln) vs incubation time relationship.

in vitro t_{1/2}=0.693/k, where k=−[slope of linear regression of % parent remaining(ln) vs incubation time]. Data analysis was performed using Microsoft Excel Software.

The results of these experiments are depicted in Table 5.

TABLE 5
Stability of Compounds in Human Liver Microsomes.

| Compound | t_{1/2} (min) | % Difference^{a} |
|---|---|---|
| Erlotinib | 57.8 | na |
| 120 | 68.5 | +19 |
| 121 | 50.8 | −12 |
| 138 | 88.5 | +53 |
| 135 | 47.1 | −19 |

^{a}% Difference = [(deuterated species) − (nondeuterated species)] (100)/(nondeuterated species)

Under the assay conditions tested (0.5 mg/mL HLM, 1 μM test compound)

Compound 120 demonstrated a 19% longer t_{1/2} compared to erlotinib, while Compound 138 demonstrated a 53% longer t_{1/2} compared to erlotinib.

### Example 16

Evaluation of Compound Stability in Human Liver Microsomes. Comparison of Compounds 105, 106, 105-H, 106-H and Erlotinib.

Example 16 is similar in design to Example 15, except that Compounds 105 and 106 were studied relative to their non-deuterated analogs and to Erlotinib. Compound 105-His the hydrogen or non-deuterated version of Compound 105 and 106-His the hydrogen or non-deuterated version of Compound 106 (i.e., in Compounds 105-H and 106-H, each Y and each Z is hydrogen).

The results of these experiments are depicted in Table 6.

TABLE 6
Stability of Compounds 105 and 106 vs 105-H, 106-H
and Erlotinib in Human Liver Microsomes.

| Compound | t_{1/2} (min) | % Difference^{a} |
|---|---|---|
| Erlotinib | 59.8 | na |
| 105 | 71.1 | 35^{b} (19)^{c} |
| 105-H | 52.6 | |
| 106 | 92.5 | 22^{b} (55)^{c} |
| 106-H | 76.0 | |

^{a}% Difference = [(deuterated species) − (nondeuterated species)] (100)/(nondeuterated species)
^{b}% Difference with respect to non-deuterated analog
^{c}% Difference with respect to non-deuterated Erlotinib

Under the assay conditions tested (0.5 mg/mL HLM, 1 μM test compound) Compound 105 demonstrated a 19% longer t_{1/2} compared to erlotinib and a 35% longer t_{1/2} compared to 105-H, while Compound 106 demonstrated a 55% longer t_{1/2} compared to erlotinib and a 22% longer t_{1/2} compared to 106-H.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of the Formula Q: 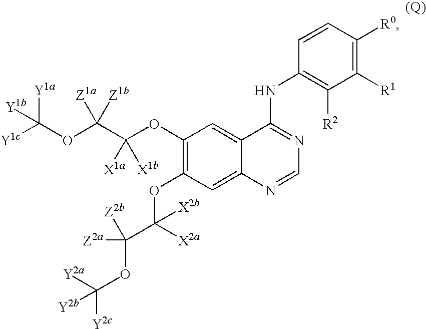
or a pharmaceutically acceptable salt thereof, wherein:
each X, each Y, and each Z is independently selected from hydrogen and deuterium;
R⁰ is selected from hydrogen, halo, —OH, —OCD₃, and —OCH₃;
R¹ is selected from —C≡CH, and —C≡CD; and
R² is selected from hydrogen and fluoro;
provided that at least one of X, Y or Z is deuterium;
further provided that when each of R⁰ and R² is hydrogen, then at least one X is deuterium; and
further provided that when each of R⁰ and R² is hydrogen, R¹ is —C≡CH, and each X and each Z is deuterium, then at least one Y is deuterium.

2. The compound of claim 1, wherein each Y¹ is the same, each Y² is the same, each Z¹ is the same, each Z² is the same, each X¹ is the same, and each X² is the same.

3. The compound of claim 2, wherein each Y¹ is deuterium, each Y² is deuterium, each Z¹ is deuterium, and each Z² is deuterium.

4. The compound of claim 1, wherein R⁰ is halo.

5. The compound of claim 1, wherein each Y¹ is deuterium, each Y² is deuterium, each Z¹ is deuterium, each Z² is deuterium, each X¹ is hydrogen, each X² is hydrogen, R¹ is —C≡CH, R² is hydrogen, and R⁰ is Br or F.

6. A compound of Formula A: 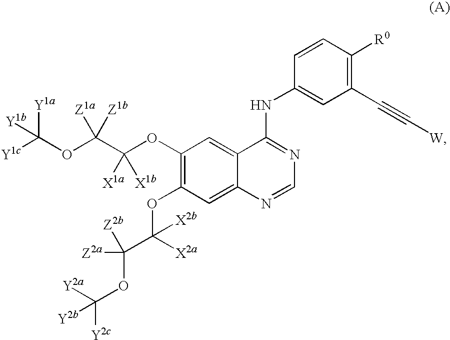
or a pharmaceutically acceptable salt thereof, wherein:
each X, each Y, each Z and W is independently selected from hydrogen and deuterium; and
R⁰ is hydrogen, OH, F, OCD₃, or OCH₃;
provided that at least one of X, Y or Z is deuterium: and
provided that if R⁰ is hydrogen, then at least one X is deuterium; and
further provided that if R⁰ is hydrogen, W is hydrogen, and each X and each Z is deuterium, then at least one Y is deuterium.

7. The compound of claim 6 selected from any one of the compounds set forth below:

| | | | | Each | Each | Each | | |
|---|---|---|---|---|---|---|---|---|
| Cmpd | Each Y¹ | Each Y² | Each Z¹ | Z² | X¹ | X² | W | R⁰ |
| 120 | D | D | D | D | D | D | H | H |
| 121 | D | D | D | D | D | D | H | F |
| 122 | D | D | D | D | D | D | H | OCH₃ |
| 123 | D | D | D | D | D | D | H | OCD₃ |
| 124 | D | D | D | D | D | D | H | OH |
| 125 | D | D | D | D | D | H | H | H |
| 126 | D | D | D | D | D | H | H | F |
| 127 | D | D | D | D | D | H | H | OCH₃ |
| 128 | D | D | D | D | D | H | H | OCD₃ |
| 129 | D | D | D | D | D | H | H | OH |
| 130 | D | D | D | D | H | D | H | H |
| 131 | D | D | D | D | H | D | H | F |
| 132 | D | D | D | D | H | D | H | OCH₃ |
| 133 | D | D | D | D | H | D | H | OCD₃ |
| 134 | D | D | D | D | H | D | H | OH |

8. The compound of claim 6 selected from any one of the compounds set forth below:

| | | | | Each | Each | Each | | |
|---|---|---|---|---|---|---|---|---|
| Cmpd | Each Y¹ | Each Y² | Each Z¹ | Z² | X¹ | X² | W | R⁰ |
| 135 | D | D | D | D | H | H | H | F |
| 136 | D | D | D | D | H | H | H | OCH₃ |
| 137 | D | D | D | D | H | H | H | OCD₃ |
| 138 | D | D | D | D | H | H | H | OH |

9. A compound of claim 6 selected from: 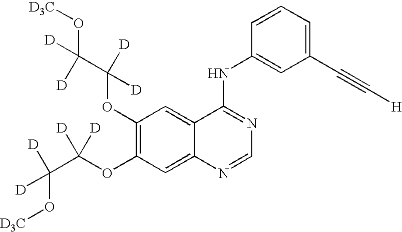
Compound 120, and

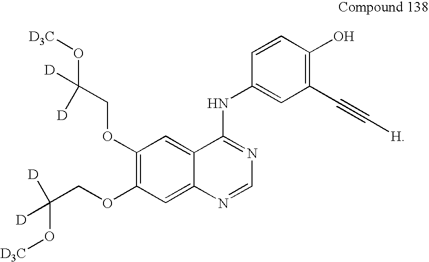

10. A compound of Formula R: 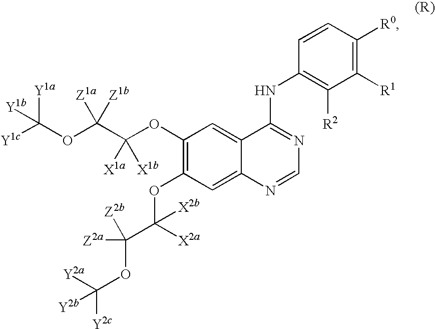
or a pharmaceutically acceptable salt thereof, wherein:
each X, each Y, and each Z is independently selected from hydrogen and deuterium;
R⁰ is selected from hydrogen, halo, —OH, —OCD₃, and —OCH₃;
R¹ is selected from hydrogen, halo, and —CF₃; and
R² is selected from hydrogen and fluoro;
provided that at least one X, Y or Z is deuterium.

11. The compound of claim 10, wherein each Y¹ is the same, each Y² is the same, each Z¹ is the same, each Z² is the same, each X¹ is the same, and each X² is the same.

12. The compound of claim 11, wherein each Y¹ is deuterium, each Y² is deuterium, each Z¹ is deuterium, and each Z² is deuterium.

13. The compound of claim 10, wherein R⁰ is hydrogen or halo.

14. The compound of claim 10 selected from any one of the compounds set forth below:

| | | | Each | Each | Each | Each | | | |
|---|---|---|---|---|---|---|---|---|---|
| Cmpd | Each Y¹ | Each Y² | Z¹ | Z² | X¹ | X² | R⁰ | R¹ | R² |
| 105 | D | D | D | D | H | H | H | Br | H |
| 106 | D | D | D | D | H | H | H | Cl | H |
| 107 | D | D | D | D | H | H | H | F | H |
| 108 | D | D | D | D | H | H | H | CF₃ | H |
| 109 | D | D | D | D | H | H | Br | Br | H |
| 110 | D | D | D | D | H | H | Br | Cl | H |
| 111 | D | D | D | D | H | H | Br | F | H |
| 112 | D | D | D | D | H | H | Br | CF₃ | H |
| 114 | D | D | D | D | H | H | F | Br | H |
| 115 | D | D | D | D | H | H | F | Cl | H |
| 116 | D | D | D | D | H | H | F | F | H |
| 117 | D | D | D | D | H | H | F | CF₃ | H |
| 119 | D | D | D | D | H | H | Br | H | F |

15. The compound of claim 14 selected from:

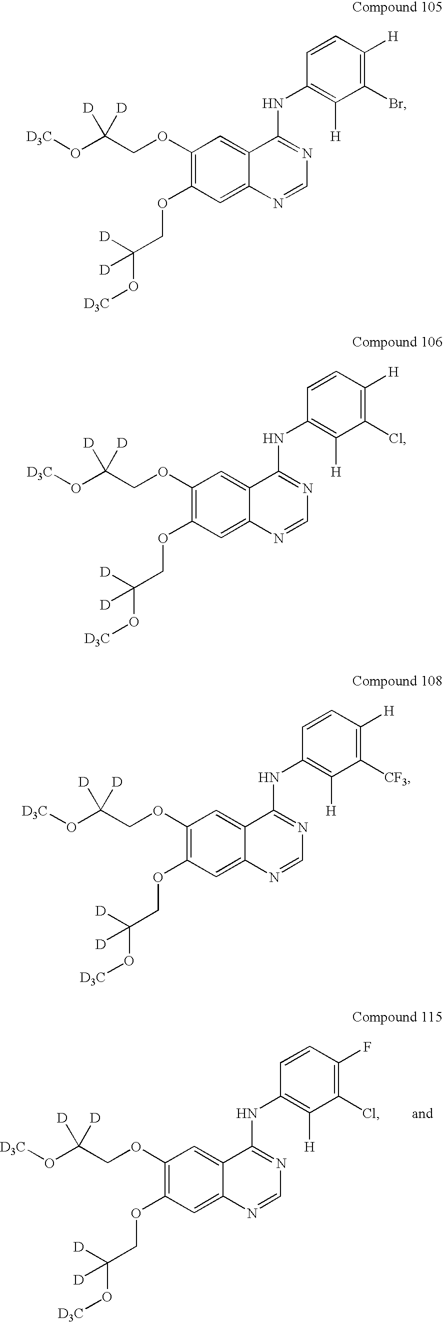

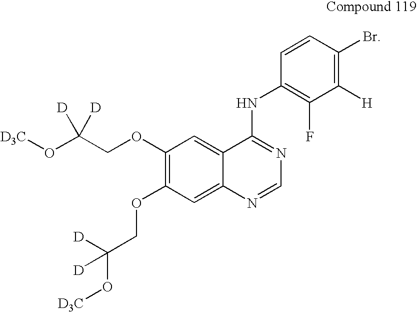

16. A pyrogen-free pharmaceutical composition comprising a compound of claim 1, and a pharmaceutically acceptable carrier.

17. The composition of claim 16, further comprising a second therapeutic agent useful in the treatment of a disease or disorder selected from cancer, inflammation, angiogenesis, vascular restenosis, immunological disorder, pancreatitis, kidney disease, blastocyte maturation and implantation, psoriasis, and benign prostatic hypertrophy (BPH).

18. The composition of claim 17, wherein the second therapeutic agent is selected from 2-deoxy-2-[¹⁸F]fluoro-D-glucose, 3′-deoxy-3′-[¹⁸F]fluorothymidine, 5-fluorouracil, AV412, avastin, bevacizumab, bexarotene, bortezomib, calcitriol, canertinib, capecitabine, carboplatin, celecoxib, cetuximab, CHR-2797, cisplatin, dasatinib, digoxin, enzastaurin, etoposide, everolimus, fulvestrant, gefitinib, gemcitabine, genistein, imatinib, irinotecan, lapatinib, lenalidomide, letrozole, leucovorin, matuzumab, oxaliplatin, paclitaxel, panitumumab, pegfilgrastim, pegylated alfa-interferon, pemetrexed, Polyphenon® E, satraplatin, sirolimus, sorafenib, sutent, sulindac, sunitinib, taxotere, temodar, temozolomide, temsirolimus, TG01, tipifarnib, trastuzumab, valproic acid, vinflunine, volociximab, vorinostat, and XL647.

19. The composition of claim 18, wherein the second therapeutic agent is bevacizumab.

20. A method of treating a patient suffering from or susceptible to a disease or disorder selected from cancer, inflammation, angiogenesis, vascular restenosis, immunological disorder, pancreatitis, kidney disease, blastocyte maturation and implantation, psoriasis, and benign prostatic hypertrophy (BPH), comprising the step of administering to the patient in need thereof a composition of claim 16.

21. The method of claim 20, wherein the patient is suffering from or susceptible to a cancer selected from non-small cell lung cancer, ovarian cancer, colorectal cancer, head and neck cancer, brain cancer, bladder cancer, sarcoma, prostate cancer, melanoma, cervical cancer, solid tumors, astrocytoma, breast cancer, pancreatic cancer, glioblastoma multiform, renal cancer, digestive/gastrointestinal cancer, liver cancer, gynecological cancers, CNS tumors, thymoma, and gastric cancer.

22. The method of claim 21, wherein the patient is suffering from non-small cell lung cancer.

23. The method of claim 22, comprising the further step of co-administering to the patient in need thereof a second therapeutic agent useful in the treatment of a disease or disorder selected from cancer, inflammation, angiogenesis, vascular restenosis, immunological disorder, pancreatitis, kidney disease, blastocyte maturation and implantation, psoriasis, and benign prostatic hypertrophy (BPH).

24. The method of claim 23, wherein the patient is suffering from cancer and the second therapeutic agent is selected from 2-deoxy-2-[¹⁸F]fluoro-D-glucose, 3′-deoxy-3′-[¹⁸F]fluorothymidine, 5-fluorouracil, AV412, avastin, bevacizumab, bexarotene, bortezomib, calcitriol, canertinib, capecitabine, carboplatin, celecoxib, cetuximab, CHR-2797, cisplatin, dasatinib, digoxin, enzastaurin, etoposide, everolimus, fulvestrant, gefitinib, gemcitabine, genistein, imatinib, irinotecan, lapatinib, lenalidomide, letrozole, leucovorin, matuzumab, oxaliplatin, paclitaxel, panitumumab, pegfilgrastim, pegylated alfa-interferon, pemetrexed, Polyphenon® E, satraplatin, sirolimus, sorafenib, sutent, sulindac, sunitinib, taxotere, temodar, temozolomide, temsirolimus, TGO1, tipifarnib, trastuzumab, valproic acid, vinflunine, volociximab, vorinostat, and XL647.

25. The method of claim 24, wherein the second therapeutic agent is bevacizumab, and the patient is suffering from non-small cell lung cancer.
